(19) United States
(12) Patent Application Publication
(10) Pub. No.: US 2011/0070135 A1
(43) Pub. Date: Mar. 24, 2011

(54) Processes and Apparatuses for Purifying Greenhouse Gases

(75) Inventors: Maria Balmas, Hacienda Heights, CA (US); Henry Chan, Fountain Valley, CA (US); Chris Gibson, Guildford (GB); Craig Skinner, Newport Beach, CA (US); Kam Vakil, Sugar Land, TX (US)

(73) Assignee: HYDROGEN ENERGY INTERNATIONAL LIMITED, Surrey (GB)

(21) Appl. No.: 12/562,263

(22) Filed: Sep. 18, 2009

(51) Int. Cl. B01D 53/62; B01D 47/00; B01D 53/46; C01B 31/20; C07C 29/00; C01C 1/04; C07C 5/02; B01J 19/00

(52) U.S. Cl. 423/220; 422/168; 423/210; 423/437.1; 568/840; 423/237; 585/899; 422/187; 422/148

(57) ABSTRACT

This invention relates to processes and apparatuses for purifying greenhouse gases. This invention relates to processes and apparatuses for drying carbon dioxide and/or producing syngas. The process includes the step of contacting a greenhouse gas stream having a contaminant with a solvent stream to at least partially reduce an amount of the contaminant and form a used solvent stream. The process includes the step of using the greenhouse gas stream for carbon sequestration, enhanced oil recovery, industrial gas supply, or chemical synthesis and production.

### BACKGROUND

1. Technical Field

This invention relates to processes and apparatuses for purifying greenhouse gases. This invention relates to processes and apparatuses for drying carbon dioxide and/or producing syngas.

2. Discussion of Related Art

Issues of greenhouse gas levels and climate change have led to development of technologies seeking to reduce and/or eliminate carbon emissions to the atmosphere. Electrical power plants that emit greenhouse gases have large capital expenditures, large maintenance expenses, high operating costs, complex operations, and generate waste streams. As technologies advance to reduce greenhouse gas emissions, various techniques to remove one or more impurities from gas streams have been developed, such as using membranes, or molecular sieves.

However, even with the above advances in purification techniques, there still remains a need and a desire for additional processes and apparatuses to purify and dry greenhouse gases, such as carbon dioxide. There also remains a need and a desire for energy production and industrial product manufacturing with reduced emissions of the greenhouse gases.

### SUMMARY

This invention relates to processes and apparatuses for purifying greenhouse gases. This invention relates to processes and apparatuses for drying carbon dioxide and/or producing syngas. Methods and uses for greenhouse gas mitigation may employ streams with reduced impurities. The plants with this invention can have reduced capital expenditures (avoiding a separate dehydration unit, for example), lower maintenance expenses (less equipment, for example), lower operating costs (same solvent as used in an acid gas recovery unit, for example), less complex operations, and/or reduced waste streams than conventional plants. Additional benefits of the processes and apparatuses of this invention may include purification and/or drying of greenhouse gases, such as carbon dioxide, along with energy production and/or industrial product manufacturing having reduced emissions of the greenhouse gases.

According to a first embodiment, this invention includes a process for purifying greenhouse gases. The process includes the step of contacting a greenhouse gas stream having a contaminant with a solvent stream to at least partially reduce an amount of the contaminant and form a used solvent stream. The process includes the step of using the greenhouse gas stream for carbon sequestration, enhanced oil recovery, industrial gas supply, chemical synthesis and production, and/or the like.

According to a second embodiment, this invention includes an apparatus for purifying greenhouse gases. The apparatus includes a greenhouse gas stream having a contaminant and a solvent stream. The apparatus includes a contacting unit connected to the greenhouse gas stream and the solvent stream, and a contacting unit effluent stream connected to the contacting unit with a reduced amount of the contaminant. The apparatus includes a used solvent stream connected to the contacting unit.

According to a third embodiment, this invention includes a process of drying carbon dioxide. The process includes the step of reacting a feedstock stream in a reactor unit to form a reactor unit effluent stream, and the step of converting the reactor unit effluent stream in a shift conversion unit to form a shift conversion unit effluent stream. The process includes the step of separating the shift conversion unit effluent stream in an acid gas removal unit to form a hydrogen stream, a hydrogen sulfide rich stream, and a carbon dioxide stream, and the step of recovering sulfur from the hydrogen sulfide rich stream in a sulfur recovery unit to form a sulfur stream and a sulfur recovery unit effluent stream. The process includes the step of contacting the sulfur recovery unit effluent stream in a tail gas treatment unit to form a tail gas treatment unit effluent stream having a reduced amount of hydrogen sulfide and a contaminant, and the step of contacting the tail gas treatment unit effluent stream in a contacting unit with a solvent stream to form a used solvent stream and a purified tail gas treatment unit effluent stream having carbon dioxide and a reduced amount of the contaminant. The process includes the step of combining the carbon dioxide stream and the purified tail gas treatment unit effluent stream to form a combined carbon dioxide stream, and the step of using the combined carbon dioxide stream for carbon sequestration, enhanced oil recovery, industrial gas supply, chemical synthesis and production, and/or the like.

According to a fourth embodiment, this invention includes an apparatus for producing syngas. The apparatus includes a feedstock stream, and a reactor unit connected to the feedstock stream. The apparatus includes a reactor unit effluent stream connected to the reactor unit, and a shift conversion unit connected to the reactor unit effluent stream. The apparatus includes a shift conversion unit effluent stream connected to the shift conversion unit, and an acid gas removal unit connected to the shift conversion unit effluent stream. The apparatus includes a hydrogen stream connected to the acid gas removal unit, and a hydrogen sulfide rich stream connected to the acid gas removal unit. The apparatus includes a carbon dioxide stream connected to the acid gas removal unit, and a sulfur recovery unit connected to the hydrogen sulfide rich stream. The apparatus includes a sulfur stream connected to the sulfur recovery unit, and a sulfur recovery unit effluent stream connected to the sulfur recovery unit. The apparatus includes a tail gas treatment unit connected to the sulfur recovery unit effluent stream, and a tail gas treatment effluent stream having a contaminant connected to the tail gas treatment unit. The apparatus includes a solvent stream connected to the acid gas removal unit, and a contacting unit connected to the tail gas treatment unit effluent stream and the solvent stream to reduce an amount of the contaminant. The apparatus includes a contacting unit effluent stream connected to the contacting unit, and a used solvent stream connected to the contacting unit and the acid gas removal unit. The apparatus includes a greenhouse gas unit connected to the contacting unit effluent stream for carbon sequestration, enhanced oil recovery, industrial gas supply, chemical synthesis and production, and/or the like. The greenhouse gas unit may include of one or more compressors and/or a combination of compressors and pumps.

### BRIEF DESCRIPTION OF THE DRAWINGS

The accompanying drawings, which are incorporated in and constitute a part of this specification, illustrate embodiments of the invention and, together with the description, serve to explain the features, advantages, and principles of the invention. In the drawings:

FIG. 1 schematically shows an apparatus for purifying greenhouse gases, according to one embodiment;

FIG. 2 schematically shows an apparatus for purifying greenhouse gases, according to one embodiment;

FIG. 3 schematically shows an apparatus for purifying greenhouse gases, according to one embodiment;

FIG. 4 schematically shows an apparatus for purifying greenhouse gases, according to one embodiment;

FIG. 5 schematically shows an apparatus for purifying greenhouse gases, according to one embodiment;

FIG. 6 schematically shows an apparatus for purifying greenhouse gases, according to one embodiment;

FIG. 7 schematically shows an apparatus for producing syngas, according to one embodiment;

FIG. 8 schematically shows an apparatus for producing syngas, according to one embodiment;

FIG. 9 schematically shows a reactor unit, according to one embodiment; and

FIG. 10 schematically shows an apparatus for producing syngas, according to one embodiment.

### DETAILED DESCRIPTION

This invention relates to processes and apparatuses for purifying greenhouse gases. This invention relates to processes and apparatuses for drying carbon dioxide and/or producing syngas.

According to one embodiment, this invention may include recycling methanol from an acid gas removal unit into a tail gas treating unit to dry carbon dioxide without a separate dehydration unit. Benefits of this configuration can include reduced capital expenditures for the separate dehydration unit, reduced maintenance and/or operating expense of an overall integrated gasification combined cycle plant, simplified operations, and/or reduced waste handling. The invention can include recycling methanol from the acid gas removal system (Rectisol unit) into the tail gas treating unit to dry the tail gas from the tail gas treatment unit absorber, such as to permit dried tail gas to be combined with carbon dioxide product for export.

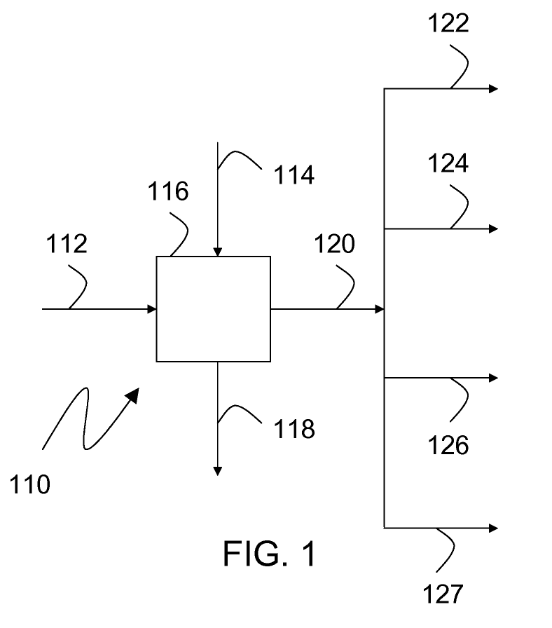

FIG. 1 schematically illustrates an apparatus 110 for purifying greenhouse gases, according to one embodiment. The apparatus 110 includes a greenhouse gas stream 112 connected to a contacting unit 116. The apparatus 110 includes a solvent stream 114 connected to the contacting unit 116. The apparatus 110 includes a used solvent stream 118 connected to the greenhouse gas stream with a reduced amount of contaminant or a contacting unit effluent stream 120. The contacting unit effluent stream 120 can be used for at least one of carbon sequestration 122, enhanced oil recovery 124, industrial gas supply 126, chemical synthesis and/or production, 127, and/or the like.

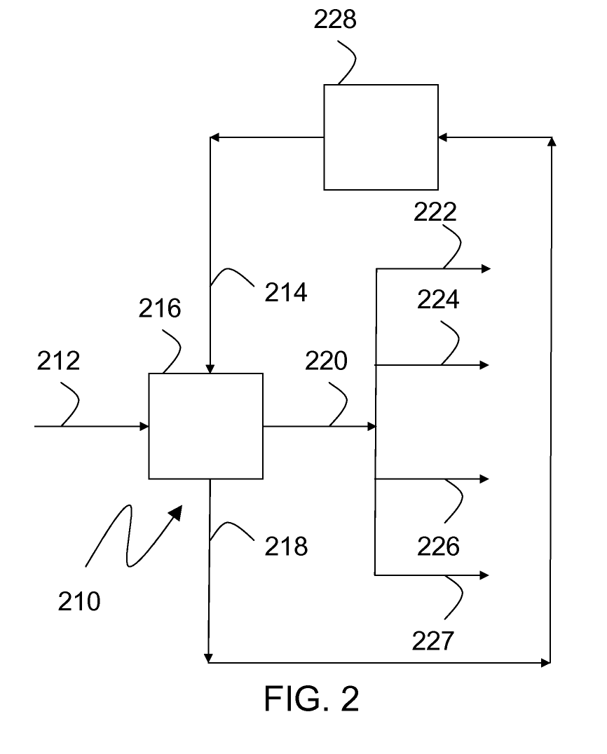

FIG. 2 schematically illustrates an apparatus 210 for purifying greenhouse gasses, according to one embodiment. The description of the apparatus 210 proceeds in accordance with the description of the apparatus 110 in FIG. 1 with changes made to the leading digit of the corresponding reference numerals. The apparatus 210 in FIG. 2 also differs from the apparatus 110 in FIG. 1 in that the apparatus 210 includes an acid gas removal unit 228 to supply the solvent stream 214 and receive the spent or used solvent stream 218.

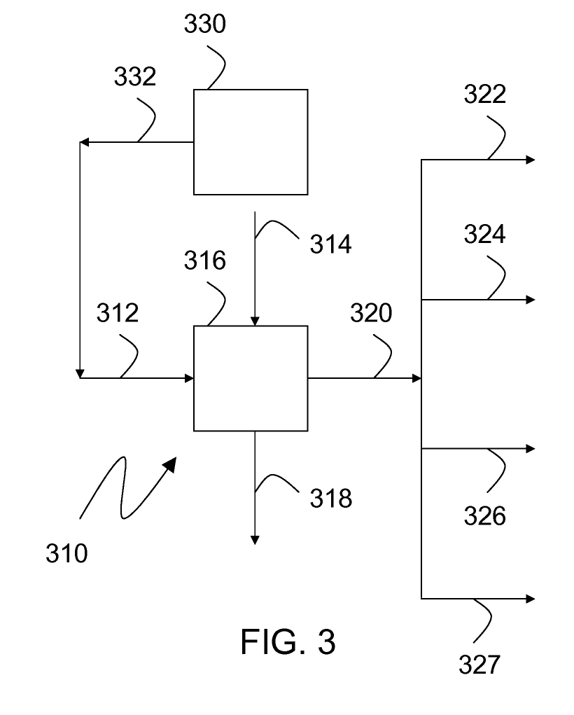

FIG. 3 schematically illustrates an apparatus 310 for purifying greenhouse gasses, according to one embodiment. The description of the apparatus, 310 proceeds in accordance with the description of the apparatus 110 in FIG. 1 with changes made to the leading digit of the corresponding reference numerals. The apparatus 310 in FIG. 3 also differs from the apparatus 110 in FIG. 1 in that the apparatus 310 includes a sulfur recovery unit 330 and a sulfur recovery unit tail gas stream 332, which forms at least a portion of the greenhouse gas stream 312.

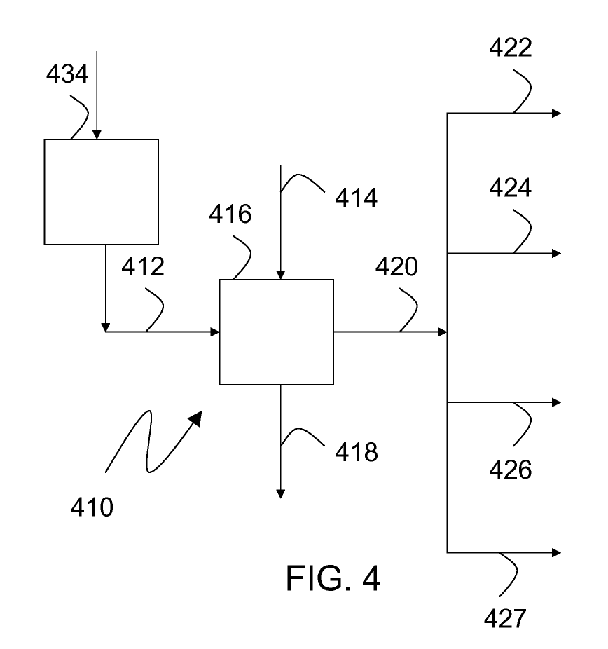

FIG. 4 schematically illustrates an apparatus 410 for purifying greenhouse gasses, according to one embodiment. The description of the apparatus 410 proceeds in accordance with the description of the apparatus 110 in FIG. 1 with changes made to the leading digit of the corresponding reference numerals. The apparatus 410 in FIG. 4 also differs from the apparatus 110 in FIG. 1 in that a tail gas treatment unit 434 processes at least a portion of the green house gas steam 412 before the contacting unit 416.

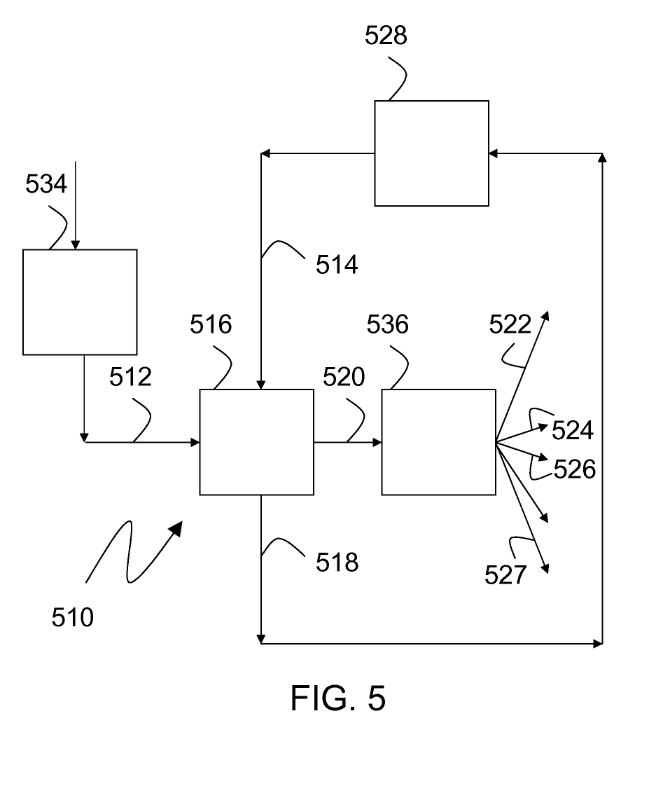

FIG. 5 schematically illustrates an apparatus 510 for purifying greenhouse gasses, according to one embodiment. The description of the apparatus 510 proceeds in accordance with the description of the apparatus 110 in FIG. 1 with changes made to the leading digit of the corresponding reference numerals. The apparatus 510 in FIG. 5 also differs from the apparatus 110 in FIG. 1 in that a tail gas treatment unit 534 processes at least a portion of the green house gas steam 512 before the contacting unit 516. The apparatus 510 also includes a recycle compression unit 536 on the contacting unit effluent stream 520 before the carbon sequestration 522, enhanced oil recovery 524, industrial gas supply 526, chemical synthesis and/or production 527, and/or the like. The apparatus 510 also includes an acid gas removal unit 528 to supply the solvent stream 514 and receive the spent or used solvent stream 518.

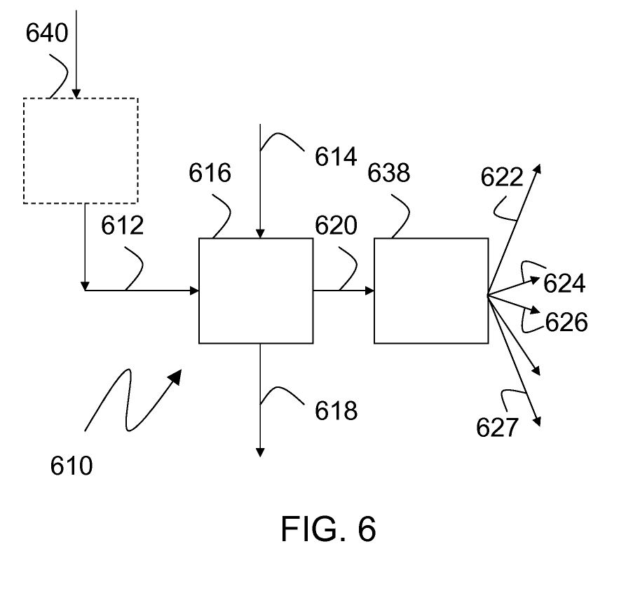

FIG. 6 schematically illustrates an apparatus 610 for purifying greenhouse gasses, according to one embodiment. The description of the apparatus 610 proceeds in accordance with the description of the apparatus 110 in FIG. 1 with changes made to the leading digit of the corresponding reference numerals. The apparatus 610 in FIG. 6 also differs from the apparatus 110 in FIG. 1 in that the apparatus 610 also includes a compression unit 638 on the contacting unit effluent stream 620 before the carbon sequestration 622, enhanced oil recovery 624, industrial gas supply 626, chemical synthesis and/or production 627, and/or the like. The apparatus also optionally includes a booster compressor or pump 640 on the green house gas stream 612.

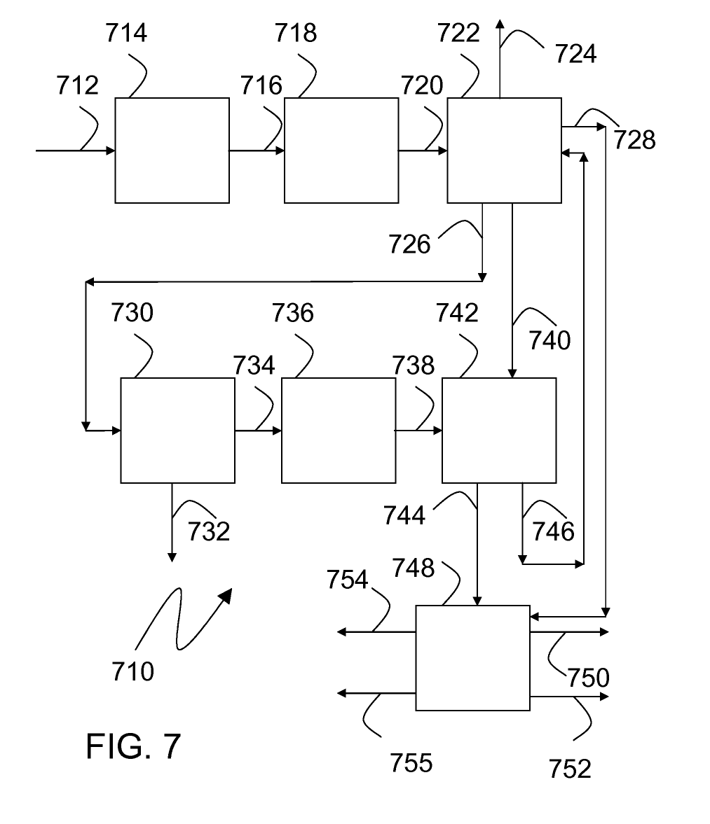

FIG. 7 schematically shows an apparatus 710 for producing syngas and/or drying carbon dioxide, according to one embodiment. The apparatus 710 includes a feedstock stream 712 connected to a reactor unit 714 with a reactor unit effluent stream 716. The reactor unit effluent stream 716 connects to a shift conversion unit 718 with a shift conversion unit effluent stream 720. Optionally, the reactor unit 714 can have one or more unshifted and/or unconverted streams, such as for use in methanol, synthetic natural gas, Fischer Trophsch liquids, and/or the like. Unshifted steams may include a suitable amount of carbon monoxide. The shift conversion unit effluent stream 720 connects to an acid gas removal unit 722 with a hydrogen stream 724, a hydrogen sulfide rich stream 726, a carbon dioxide stream 728, and optionally a chemical stream (not shown). The chemical stream may be used with an unshifted stream. The carbon dioxide stream 728 connects to a greenhouse gas unit 748. The hydrogen sulfide rich stream 726 connects to a sulfur recovery unit 730 with a sulfur stream 732 and a sulfur recovery unit effluent stream 734. The sulfur recovery unit effluent stream 734 connects to a tail gas treatment unit 736 with a tail gas treatment effluent stream 738. The acid gas removal unit 722 supplies a solvent stream 740 to a contacting unit 742 with a contacting unit effluent stream 744 and a used solvent stream 746. The tail gas treatment effluent stream 738 also connects to the contacting unit 742. The contacting unit effluent stream 744 connects to a greenhouse gas unit 748 for at least one of carbon sequestration 750, enhanced oil recovery 752, industrial gas supply 754, chemical synthesis and/or production 755, and/or the like.

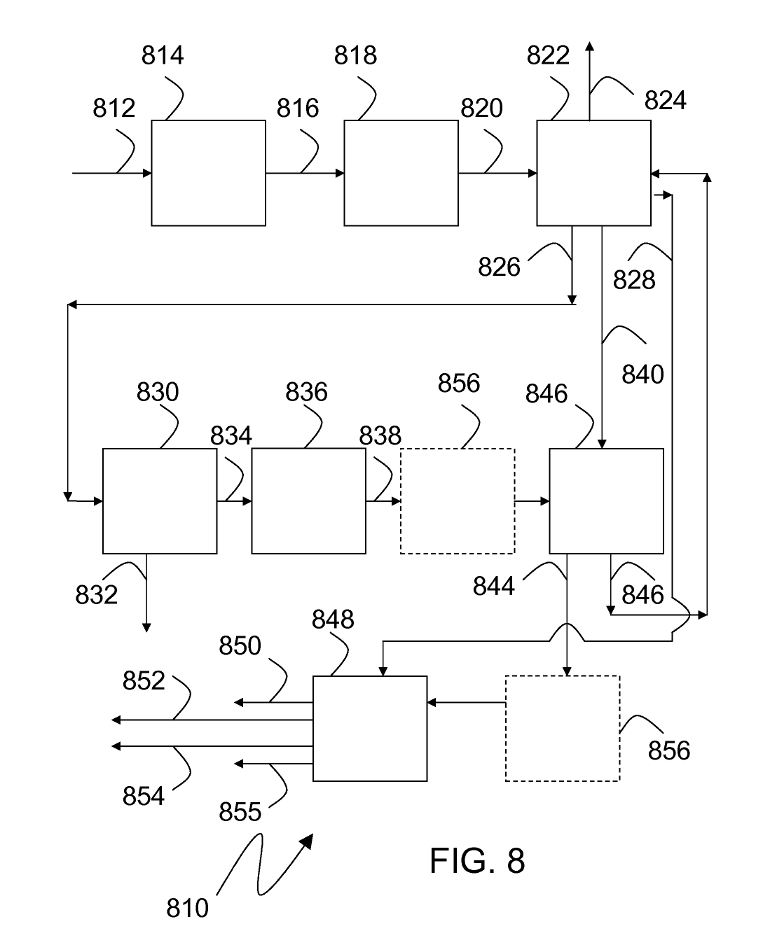

FIG. 8 schematically illustrates an apparatus 810 for producing syngas and/or drying carbon dioxide, according to one embodiment. The description of the apparatus 810 proceeds in accordance with the description of the apparatus 710 in FIG. 7 with changes made to the leading digits of the corresponding reference numerals. The apparatus 810 in FIG. 8 also differs from the apparatus 710 in FIG. 7 in that the apparatus 810 also includes a compression unit 856 on the tail gas treatment unit effluent stream 838 or the contacting unit effluent stream 844.

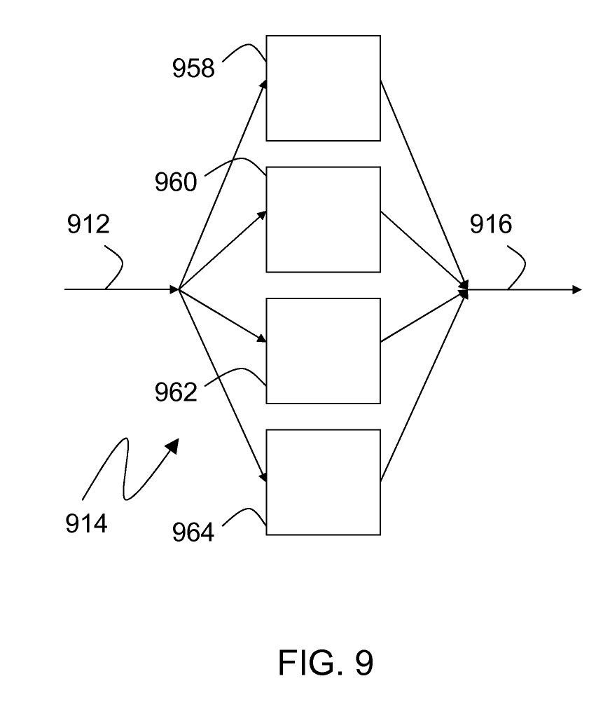

FIG. 9 schematically shows a reactor unit 914, according to one embodiment. The reactor unit 914 receives a feedstock stream 912 and produces a reactor unit effluent stream 916. The reactor unit 914 may include at least one of a gasification unit 958, a reforming unit 960, a partial oxidation unit 962, a pyrolysis unit 964, and/or the like.

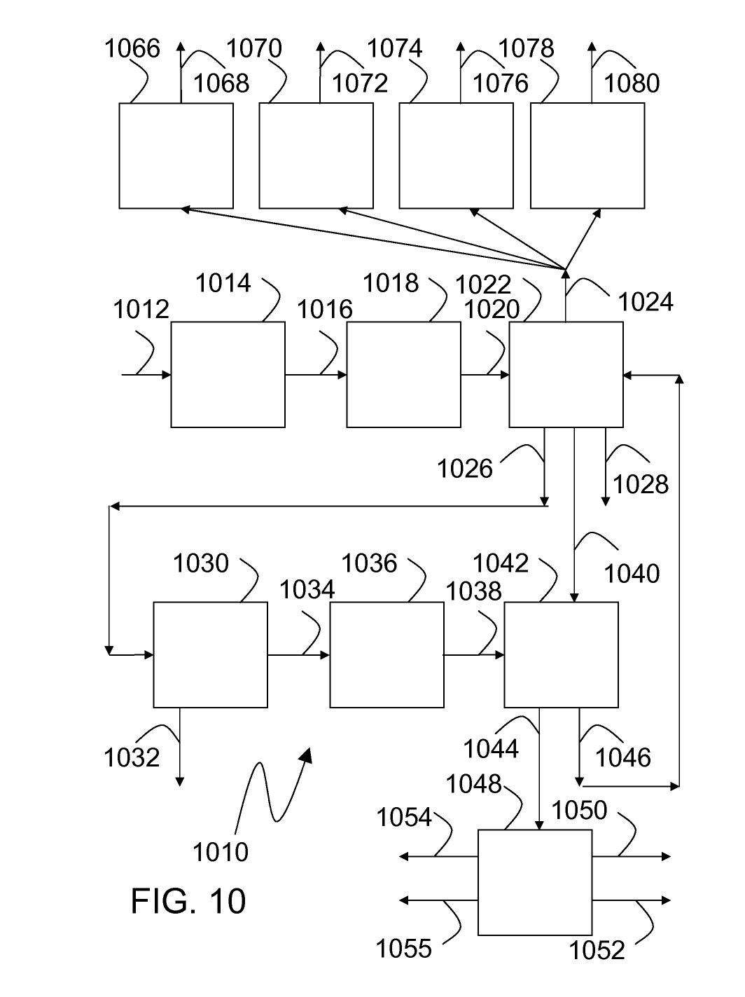

FIG. 10 schematically shows an apparatus 1010 for producing syngas and/or drying carbon dioxide, according to one embodiment. The description of the apparatus 1010 proceeds in accordance with the description of the apparatus 710 in FIG. 7 with changes made to the leading digits of the corresponding reference numerals. The apparatus 1010 in FIG. 10 also differs from the apparatus 710 in FIG. 7 in that the apparatus 1010 also includes on the hydrogen stream 1024 at least one of an electricity generation unit 1066 with an electricity stream 1068, an ammonia generation unit 1070 with an ammonia stream 1072, a methanol generation unit 1074 with a methanol stream 1076, a hydrocarbon product generation unit 1078 with a hydrocarbon product stream 1080, and/or the like.

According to one embodiment, the invention may include a process for purifying greenhouse gases. The process may include the step of contacting a greenhouse gas stream having a contaminant with a solvent stream to at least partially reduce an amount of the contaminant and form a used solvent stream. The process may include the step of using the greenhouse gas stream for at least one of carbon sequestration, enhanced oil recovery, industrial gas supply, chemical synthesis and production, and/or the like.

Process broadly refers to a proceeding, a series of events and/or steps, progress and/or the like, such as to accomplish a task, a goal, and/or an outcome. Processes may be batch, semi-batch, discrete, continuous, semi-continuous, and/or the like.

Purify broadly refers to removing, reducing, and/or eliminating a defilement, a contaminant, an imperfection, an undesirable element, and/or the like. Purifying can remove any suitable amount of a contaminant, such as by at least about 10 percent, at least about 25 percent, at least about 50 percent, at least about 75 percent, at least about 90 percent, at least about 95 percent, at least about 99 percent, and/or the like of the contaminant from an incoming stream on a mass basis, a volume basis, a mole basis, and/or the like.

Gas broadly refers to not being primarily in a solid state and/or a liquid state, such as having a generally indefinite volume (compressible) and/or a generally indefinite shape (fills its container). Gases may be primarily vapors but also may include solid or particulate matter and/or fine liquid droplets, such as to form a suspension and/or an aerosol.

Greenhouse gases broadly refer to gases and/or vapors in an atmosphere that can absorb and/or emit radiation within the thermal infrared range, such as carbon monoxide, carbon dioxide, water vapor, methane, ethane, propane, ozone, hydrogen sulfide, sulfur oxides, nitrogen oxides, halocarbons, chlorofluorocarbons (CFCs), and/or the like.

Without being bound by theory, greenhouse gases are believed to receive and/or retain solar radiation and/or energy that becomes trapped in the atmosphere and causes an increase in average global atmospheric temperatures and/or climate change. According to one embodiment, the greenhouse gas includes primarily, mostly, and/or substantially carbon dioxide, such as at least about 50 percent, at least about 80 percent, at least about 90 percent, at least about 95 percent, and/or the like on a mass basis, a volume basis, a mole basis, and/or the like. The greenhouse gas can be at any suitable temperature, pressure, and/or have any suitable phase and/or state, such as a solid phase, a liquid phase, a gas phase, a supercritical fluid phase, and/or the like.

Contact broadly refers to bring into communication (fluid), join, mix, combine, and/or the like. According to one embodiment, contact includes intimate mixing of streams and/or substances. Contact may result in reaching at least a portion of thermodynamic equilibrium (flash). Contact may include a use of shear, velocities, gradients, diffusion, kinetics, other physical principles, other chemical principles, and/or the like.

Stream broadly refers to a flow, a succession, a supply, and/or the like of a material, a substance, and/or the like.

Contaminant broadly refers to unwholesome and/or undesirable elements, materials, compounds, and/or the like, such as to corrupt, soil, infect, pollute, defile, make impure, make inferior, make tainted, and/or the like. The contaminant may be in any suitable amount, such as between about zero percent and about 50 percent, between about 0.001 percent and about 20 percent, between about 0.01 percent and about 5 percent, and/or the like on a mass basis, a volume basis, a mole basis, and/or the like.

Solvent broadly refers to a substance and/or material capable at least in part of dissolving and/or dispersing one or more other materials and/or substances, such as to provide and/or form a solution. The solvent may be polar, nonpolar, neutral, protic, aprotic, and/or the like. The solvent may include any suitable element, molecule, and/or compound, such as methanol, ethanol, propanol, glycols, ethers, ketones, other alcohols, amines, salt solutions, and/or the like. The solvent may include physical solvents, chemical solvents, and/or the like. The solvent may operate by any suitable mechanism, such as physical absorption, chemical absorption, chemisorption, physisorption, adsorption, pressure swing adsorption, temperature swing adsorption, and/or the like.

At least partially reduce includes any suitable amount lowered and/or removed, such as by at least about 10 percent, at least about 25 percent, at least about 50 percent, at least about 75 percent, at least about 90 percent, at least about 95 percent, at least about 99 percent, and/or the like of the contaminant from an incoming stream on a mass basis, a volume basis, a mole basis, and/or the like.

Form broadly refers to make up, constitute, develop, give shape, and/or the like.

Used solvent stream broadly refers to at least a portion of the solvent stream having at least some amount of the contaminant. The used solvent stream may include any suitable amount of the contaminant such as between about 0.001 percent and about 50 percent, between about 0.01 percent and about 25 percent, between about 0.1 percent and about 10 percent, and/or the like on a mass basis, a volume basis, a mole basis, and/or the like.

Use broadly refers to put into action or service, to carry out a purpose, and/or the like.

Carbon sequestration broadly refers to long-term storage of carbon dioxide and/or other forms of carbon, such as by use of geoengineering techniques to deposit carbon into the ocean, land surface, and/or the like. Carbon sequestration may also include aspects of carbon capture and storage, such as injection into geologic formations. The carbon sequestration may use any suitable device and/or equipment, such as a carbon sequestration unit with compressors, pumps, and/or the like.

Enhanced oil recovery broadly refers to techniques and/or strategies to increase an amount of hydrocarbon recovered and/or removed from a geological structure. Enhanced oil recovery may include gas injection, chemical injection, ultrasonic stimulation, microbial injection, thermal recovery, and/or the like. Enhanced oil recovery may increase an amount of crude oil, natural gas, bitumen, coal, and/or the like. The enhanced oil recovery may use any suitable device and/or equipment, such as an enhanced oil recovery unit with compressors, pumps, and/or the like.

Industrial gas supply broadly includes uses and gases for commercial purposes and/or applications, such as refrigeration, food preservation, food preparation, beverage preparation, medical usage, chemical processes, biological processes, metallurgical processes, and/or the like. The industrial gas supply may use any suitable device and/or equipment, such as an industrial gas supply unit with compressors, pumps, and/or the like.

Chemical synthesis and production broadly includes materials and/or compounds derived at least in part from the streams of the processes and/or, apparatuses, such as the greenhouse gas stream and/or carbon dioxide stream. The chemical synthesis and production may use any suitable device and/or equipment, such as a chemical synthesis and production unit compressors, reactors, pumps, and/or the like. According to one embodiment, the chemicals may include urea, carbonic acid, other fertilizers, and/or the like. The chemical synthesis and production unit may include a urea unit, a carbonic acid generation unit, and/or the like.

According to one embodiment, the process further may include the step of supplying the solvent stream for contacting the greenhouse gas stream from an acid gas removal unit, and the step of returning the solvent stream after contacting with the greenhouse gas stream to the acid gas removal unit. The acid gas removal unit may be the supply and/or source of the solvent stream and the return and/or disposal of the used solvent stream. Desirably, but not necessarily, the acid gas removal unit removes at least a portion of the contaminant, such as to regenerate and/or recycle the solvent.

Unit broadly refers to a collection, a group, and/or an assembly of devices and/or equipment, such as to accomplish and/or perform a task and/or an outcome. Units may include any suitable process equipment and/or devices, such as vessels, columns, pumps, valves, compressors, control systems, and/or the like.

Acid gas removal unit broadly refers to any suitable device and/or equipment to separate at least a portion of acid gas from another process stream, such as a hydrogen stream and/or syngas stream. Acid gas broadly refers to a gas and/or vapor that contains hydrogen sulfide, carbon dioxide, other similar contaminants, and/or the like. Desirably, the acid gas removal unit can separate and/or form a hydrogen stream or a purified syngas stream, and an acid gas stream. The acid gas removal unit may also separate the acid gas stream into one or more components and/or constituents, such as into a carbon dioxide stream and a hydrogen sulfide stream.

The acid gas removal unit may include any suitable device and/or equipment, such as pumps, valves, pipes, compressors, heat exchangers, pressure vessels, distillation columns, control systems, and/or the like. According to one embodiment, the acid gas removal unit includes one or more absorber towers and one or more stripper towers. The acid gas removal unit may recover and/or separate any suitable amount of acid gas from a process stream, such as at least about 50 percent, at least about 75 percent, at least about 85 percent, at feast about 90 percent, at least about 95 percent, at least about 99 percent, and/or the like on a mass basis, a volume basis, a mole basis, and/or the like.

The acid gas removal unit may include Rectisol systems from Linde AG, Munich, Germany, and/or Lurgi GmbH, Frankfurt, Germany, Selexol systems from UOP, Des Plaines, Illinois, U.S.A., methanol systems, alcohol systems, amine systems, promoted amine systems, hindered amine systems, glycol systems, ether systems, potassium carbonate systems, water scrubbing systems, other suitable solvents, and/or the like.

According to one embodiment, the solvent stream includes primarily methanol. The solvent stream may be at any suitable pressure and/or temperature, such as a temperature of between about 40 degrees Celsius and about −100 degrees Celsius, between about 20 degrees Celsius and about −80 degrees Celsius, between about 0 degrees Celsius and about −60 degrees Celsius, and/or the like prior to contacting the greenhouse gas stream.

According to one embodiment, a sulfur recovery unit tail gas steam supplies at least a portion of the greenhouse gas stream. Sulfur recovery unit broadly refers to any suitable device and/or equipment to convert at least a portion of sulfur containing compounds into elemental sulfur and/or other suitable materials, such as converting hydrogen sulfide into molten elemental sulfur. Recovering elemental sulfur may include any suitable process and/or chemical reaction, such as such as converting hydrogen sulfide into molten elemental sulfur. Elemental broadly refers to relating to being primarily an element. Elemental states may include any suitable form, such as amorphous forms, crystalline forms, solid forms, liquid forms, and/or the like. Recovering sulfur in other forms and/or compounds is within the scope of this invention.

The sulfur recovery process may any suitable include reactions, such as those used in a Claus unit like oxidation, decomposition, forming pollutants, and/or the like. The sulfur recovery process may include any suitable device and/or equipment, such as with one or more burners, one or more condensers, one or more catalyst beds, and/or the like. The sulfur recovery unit may convert any suitable portion of hydrogen sulfide in a feed stream to elemental sulfur, such as at least about 50 percent, at least about 75 percent, at least about 85 percent, at least about 90 percent, at least about 95 percent, at least about 99 percent, and/or the like on a mass basis, a volume basis, a mole basis, and/or the like.

Tail gas broadly refers to an exit stream and/or an exhaust from a unit and/or device. The tail gas may be at any suitable temperature and/or pressure. The tail gas may be vented to atmosphere, used in subsequent processing, used in subsequent pollution control devices, used in subsequent heat recovery, used in subsequent power recovery, and/or the like.

The sulfur recovery unit effluent tail gas stream may supply any suitable amount of the greenhouse gas stream, such as about 100 percent, between about 10 percent and about 95 percent, and/or the like on a mass basis, a volume basis, a mole basis, and/or the like.

Effluent broadly refers to flowing out of, leaving and/or exiting.

According to one embodiment, the process may include the step of treating and/or processing the greenhouse gas stream in a tail gas treatment unit before contacting with the solvent.

The contaminant may include any suitable material and/or substance, such as primarily water.

The greenhouse gas stream with a reduced amount of the contaminant may include any suitable moisture content, such as a dew point of between about 20 degrees Celsius and about −80 degrees Celsius, between about 0 degrees Celsius and about −60 degrees Celsius, between about −20 degrees Celsius and about −40 degrees Celsius, and/or the like.

The invention has been generally described with integrated use of acid gas removal solvent for drying carbon dioxide, but embodiments with separate (independent) and/or different solvent streams and/or dehydration systems are within the scope of this invention.

According to one embodiment, the invention may include an apparatus for purifying greenhouse gases. The apparatus may include a greenhouse gas stream having a contaminant, and a solvent stream. The apparatus may include a contacting unit connected to the greenhouse gas stream and the solvent stream, and a contacting unit effluent stream connected to the contacting unit with a reduced amount of the contaminant. The apparatus may include a used solvent stream connected to the contacting unit.

Apparatus broadly refers to one or more devices and/or equipment to perform and/or accomplish a step, a task, and/or an outcome. Apparatuses may use mechanical principles, chemical principles, thermodynamic principles, and/or the Like. The apparatus and any parts and/or portions of the apparatus may have any of the features and/or characteristics with respect to processes and/or apparatuses described within this specification.

Device broadly refers to a piece of equipment and/or a mechanism, such as to perform and/or accomplish a step, a task, and/or an outcome. One or more devices may form a portion of a unit and/or an apparatus.

Connect broadly refers to join and/or establish communication, such as fluid communication. Fluid communication (flow) may be established by any suitable manner, such as pipes, tubing, conduits, channels, flow paths, placing in proximity, and/or the like. Connecting may include any suitable motive force devices, such as to move a substance and/or a material from one location to another. Motive force devices may include pumps, compressors, blowers, ejectors, eductors, conveyors, and/or the like.

The contacting unit may include any suitable equipment and/or structure, such as mixing valves, static mixers, trays, random packing, structured packing, demisters, and/or the like. The contacting unit may include one or more pressure vessels operating in series and/or parallel configurations.

According to one embodiment, the apparatus may further include a sulfur recovery unit tail gas treatment unit connected to the greenhouse gas stream to supply at least a portion of the greenhouse gas stream. The apparatus may further include an acid gas removal unit for supplying the solvent stream and receiving the used solvent stream. The apparatus may further include a recycle compression unit connected to the contacting unit effluent stream for at least one of carbon sequestration, enhanced oil recovery, industrial gas supply, chemical synthesis and production, and/or the like.

The sulfur recovery unit tail gas treatment unit may include any suitable devices and/or equipment, such as a burner, a catalyst bed, an ammonia scrubber, a brine treatment device, an amine contactor, a wash column, a regeneration column and/or the like. The tail gas treatment unit can reduce hydrogen sulfides, carbonyl sulfide, sulfur oxides and/or the like.

Recycle compression unit broadly refers to any suitable compression devices, such as centrifugal compressors, screw compressors, positive displacement compressors, reciprocating compressors, and/or the like. The compressors may include one or more stages operating in series and/or parallel configurations. The recycle compression unit can increase a pressure of the stream to any suitable value, such as below about vacuum, at least about 1 bar absolute, at least about 3 bar absolute, at least about 5 bar absolute, at least about 10 bar absolute, and/or the like. The recycle compression unit may liquefy at least a portion of a stream, such as to form liquid carbon dioxide. The recycle compression unit may form a supercritical fluid (above the critical point), such as to form supercritical carbon dioxide.

According to one embodiment, the apparatus further may include a compression unit on the greenhouse gas stream and/or the contacting unit effluent stream. The compression unit may include a single stage device, a multistage device, and/or the like. Optionally, a booster compression device and/or a pump may be on the green house gas stream. The compression unit may have any of the features and/or characteristics of the recycle compression unit described in this specification.

Optionally broadly refers to being not compulsory and/or needed, such as with an act of choosing. Optionally may include periodic and/or cyclic operations in addition to continuous operations.

According to one embodiment, the apparatus may include where the greenhouse gas stream may include primarily carbon dioxide, the solvent stream may include primarily methanol, and/or the contaminant may include primarily water.

According to one embodiment, the apparatus may include where the solvent stream has a temperature of between about 20 degrees Celsius and about −80 degrees Celsius, and the greenhouse gas effluent stream with a reduced amount of the contaminant after the contacting unit has a dew point of between about 0 degrees Celsius and about −60 degrees Celsius.

According to one embodiment, the invention may include a process of drying carbon dioxide. The process may include the step of reacting a feedstock stream in a reactor unit to form a reactor unit effluent stream, and the step of converting the reactor unit effluent stream in a shift conversion unit to form a shift conversion unit effluent stream. The process may include the step of separating the shift conversion unit effluent stream in an acid gas removal unit to form a hydrogen stream, a hydrogen sulfide stream, and a carbon dioxide stream, and the step of recovering sulfur from the hydrogen sulfide stream in a sulfur recovery unit to form a sulfur stream and a sulfur recovery unit effluent stream. The process may include the step of contacting the sulfur recovery unit effluent stream in a tail gas treatment unit to form a tail gas treatment unit effluent stream with a reduced amount of hydrogen sulfide and a contaminant, and the step of contacting the tail gas treatment unit effluent stream in a contacting unit with a solvent stream to form a used solvent stream and a purified tail gas treatment unit effluent stream with carbon dioxide and a reduced amount of the contaminant. The process may include the step of combining the carbon dioxide stream and the purified tail gas treatment unit effluent stream to form a combined carbon dioxide stream, and the step of using the combined carbon dioxide stream for at least one of carbon sequestration, enhanced oil recovery, industrial gas supply, chemical synthesis and production, and/or the like.

Drying broadly refers to reducing and/or removing at least a portion of moisture content from a material and/or a substance. Drying may include reducing a dew point by any suitable amount, such as. at least about 10 degrees Celsius, at least about 25 degrees Celsius, at least about 40 degrees Celsius, and/or the like.

Feedstock broadly refers to any suitable material and/or substance for consumption, reaction, conversion, processing, and/or the like. According to one embodiment, the feedstock may include carbonaceous materials, such as coal, peat, coke, petroleum coke, bitumen, crude oil, tar sands, biomass, biomass char, and/or the like. Desirably, but not necessarily, at least a portion of the feedstock may originate and/or be supplied from renewable resources, such as non-fossil fuels.

Biomass broadly refers to plant and/or animal materials and/or substances derived at least in part from living substances, such as lignocellulosic sources. Lignocellulosic broadly refers to containing cellulose, hemicellulose, lignin, and/or the like, such as plant material. Lignocellulosic material may include any suitable material, such as sugar cane, sugar cane bagasse, energy cane, energy cane bagasse, rice, rice straw, corn, corn stover, wheat, wheat straw, maize, maize stover, sorghum, sorghum stover, sweet sorghum, sweet sorghum stover, cotton, cotton remnant, sugar beet, sugar beet pulp, soybean, rapeseed, jatropha, switchgrass, miscanthus, other grasses, algae, fungi, bacteria, timber, softwood, hardwood, wood bark, wood waste, sawdust, paper, paper waste, agricultural waste, manure, dung, sewage, municipal solid waste, any other suitable biomass material, and/or the like.

Reacting broadly refers to any suitable transformation with at least a portion of a chemical step, such as at least one of combustion, oxidation, partial oxidation, pyrolysis, cracking, thermal cracking, reforming, reduction, synthesis, decomposition, single replacement, double replacement, and/or the like. Reactions may be exothermic, endothermic, and/or the like. Reactions may or may not utilize a catalyst, such as to increase a reaction rate. Catalysts may be homogenous, heterogeneous, supported, unsupported, and/or the like.

According to one embodiment, the step of reacting produces at least some amount of hydrogen and/or syngas. Syngas broadly refers to a mixture of gases derived at least in part from synthetic steps and/or actions. The syngas may include any suitable composition, such as primarily hydrogen with some amount of carbon oxides (carbon monoxide and/or carbon dioxide) and/or other contaminants. The syngas may have any suitable energy content, such as high value syngas with an energy content greater than methane on a volumetric basis, syngas with an energy content about equal to methane on a volumetric basis, low value syngas with an energy content less than methane on a volumetric basis, and/or the like. The syngas may include any suitable ratio of hydrogen to carbon monoxide, such as may be useful for synthesis reactions and/or the like. Carbon monoxide content may be controlled by various manners, such as reactor unit operation, shift conversion . unit operation, shift conversion unit bypass (partial and/or complete), and/or the like. Embodiments without shift conversion units are within the scope of this invention.

The reactor unit may include any suitable equipment and/or devices, such as furnaces, reformers, combustors, gasifiers, fixed beds, fluidized beds, slurry beds, risers, downers, regenerators, heat exchangers, quenches, pressure vessels, pipes, valves, pumps, compressors, control systems, and/or the like. According to one embodiment, the reactor unit includes at least one of a gasification unit, a reforming unit, a steam methane reforming unit, an oxidation unit, a partial oxidation unit, a pyrolysis unit, a coking unit, a cracking unit, a catalytic cracking unit, a thermal cracking unit, and/or the like. The reactor unit may convert any suitable amount of the feedstock stream into hydrogen and/or syngas.

The reactor unit may form a portion of a larger plant, such as a power plant, a petroleum refinery, a chemical production complex, and/or the like. The plant may include simple cycle gas turbines, combined cycle gas turbines, heat recovery units, boilers, steam generators, and/or the like. The plant may include an integrated gasification combined cycle (IGCC) configuration optionally with carbon sequestration. Desirably, but not necessarily, the plant operates with reduced carbon emissions compared to plants of conventional configuration, such as a coal fired boiler exhausting directly to the atmosphere.

Converting broadly refers to changing from one thing and/or property into another, such as carbon monoxide into carbon dioxide. Reducing and/or lowing carbon monoxide content may include a water gas shift reaction that consumes water molecules and produces hydrogen molecules. The shift conversion unit may include one or more shift converters in series and/or parallel configurations optionally with cooling in between, such as one or more heat exchangers. The shift converters may operate in order of decreasing temperature, such as a high temperature shift converter and a low temperature shift converter. The shift converters may include any suitable catalysts, such as sweet shift catalyst, sour shift catalyst, and/or the like.

The hydrogen may be used for any suitable purpose, such as at least one of sold for industrial gas supply, sold for fuel, sold for transportation fuel, used to produce steam, used to produce electricity, used to produce ammonia, used to produce methanol, used to produce synthetic hydrocarbon products, and/or the like. Synthetic hydrocarbon products broadly refer to compounds made by gas to liquids techniques and/or the like, such as Fischer-Tropsch processes, methanol to olefins, and/or the like. Synthetic hydrocarbons may include straight chain molecules, branched molecules, saturated molecules, unsaturated molecules, cyclic molecules, aromatic molecules, and/or the like. The synthetic hydrocarbons may include any other suitable functionality, such as ethers, alcohols, ketones, and/or the like. The synthetic hydrocarbons may be suitable for fuel usage, such as gasoline, gasoline blending stock, diesel, diesel blending stock, aviation fuel, aviation fuel blending stock, heating oil, heating oil blending stock, other transportation fuels, and/or the like. In the alternative, the synthetic hydrocarbons may be suitable for other applications and/or uses, such as chemical feedstocks, chemical products, solvents, coatings, surfactants, adhesives, copolymers, fertilizers, pharmaceuticals, and/or the like. According to one embodiment, the methanol generation unit may supply at least of portion of the methanol used in the acid gas removal unit.

According to one embodiment, the process may include where the greenhouse gas stream includes primarily carbon dioxide, the solvent stream includes primarily methanol, and the contaminant includes primarily water.

According to one embodiment, the solvent stream includes a temperature of between about 20 degrees Celsius and about −80 degrees Celsius, and the purified tail gas treatment unit effluent stream includes a dew point of between about least 0 degrees Celsius and about −60 degrees Celsius.

According to one embodiment, the invention may include an apparatus for producing syngas. The apparatus may include a feedstock stream, and a reactor unit connected to the feedstock stream. The apparatus may include a reactor unit effluent stream connected to the reactor unit, and a shift conversion unit connected to the reactor unit effluent stream. The apparatus may include a shift conversion unit effluent stream connected to the shift conversion unit, and an acid gas removal unit connected to the shift conversion unit effluent stream. The apparatus may include a hydrogen stream connected to the acid gas removal unit, and a hydrogen sulfide stream connected to the acid gas removal unit. The apparatus may include a carbon dioxide stream connected to the acid gas removal unit, and a sulfur recovery unit connected to the hydrogen sulfide stream. The apparatus may include a sulfur stream connected to the sulfur recovery unit, and a sulfur recovery unit effluent stream connected to the sulfur recovery unit. The apparatus may include a tail gas treatment unit connected to the sulfur recovery unit effluent stream, and a tail gas treatment effluent stream with a contaminant connected to the tail gas treatment unit. The apparatus may include a solvent stream connected to the acid gas removal unit, and a contacting unit connected to the tail gas treatment unit effluent stream and the solvent stream to reduce an amount of the contaminant. The apparatus may include a contacting unit effluent stream connected to the contacting unit, and a used solvent stream connected to the contacting unit and the acid gas removal unit. The apparatus may include a greenhouse gas unit connected to the contacting unit effluent stream for at least one of carbon sequestration, enhanced oil recovery, industrial gas supply, chemical synthesis and production, and/or the like.

The contacting unit of the apparatus may include mixing valves, static mixers, trays, random packing, structured packing, demisters, and/or the like. The apparatus may include a compression unit on the tail gas treatment unit effluent stream and/or the contacting unit effluent stream. The apparatus may include where the tail gas treatment unit effluent stream includes primarily carbon dioxide, the solvent stream includes primarily methanol, and the contaminant includes primarily water.

The solvent may have a temperature of between about 20 degrees Celsius and about −80 degrees Celsius, and the contacting unit effluent stream may have a dew point of between about 0 degrees Celsius and about −60 degrees Celsius. The reactor unit may include at least one of a gasification unit, a reforming unit, a partial oxidation unit, a pyrolysis unit, and/or the like.

The apparatus may further include one or more of a steam generation unit, an electricity generation unit, an ammonia generation unit, a methanol generation unit, a synthetic hydrocarbon product generation unit, and/or the like. Generation broadly refers to producing, making, manufacturing, and/or the like. The generation units may use and/or consume at least a portion of the hydrogen stream. Optionally, a methantion unit may further remove carbon oxides and/or other contaminants from the hydrogen stream.

The steam generation unit may include any suitable device and/or equipment, such as heat exchangers, boilers, steam generators, turbines, condensers, and/or the like. The electricity generation unit may include any suitable device and/or equipment, such as electricity generators, transformers, and/or the like. The ammonia generation unit may include any suitable device and/or equipment, such as compressors, converters, refrigeration systems, and/or the like. The methanol generation unit may include any suitable device and/or equipment, such as compressors, converters, refrigeration systems, and/or the like. The synthetic hydrocarbon generation unit may include any suitable device and/or equipment, such as compressors, reactors, and/or the like.

As used herein the terms “has”, “having”, “comprising”, “with”, and “including” are open and inclusive expressions. Alternately, the term “consisting” is a closed and exclusive expression. Should any ambiguity exist in construing any term in the claims or the specification, the intent of the drafter is toward open and inclusive expressions.

As used herein the term “and/or the like” provides support for any and all individual and combinations of items and/or members in a list, as well as support for equivalents of individual and combinations of items and/or members.

Regarding an order, number, sequence, and/or limit of repetition for steps in a method or process, the drafter intends no implied order, number, sequence and/or limit of repetition for the steps to the scope of the invention, unless explicitly provided.

Regarding ranges, ranges are to be construed as including all points between upper values and lower values, such as to provide support for all possible ranges contained between the upper values and the lower values including ranges with no upper bound and/or lower bound.

It will be apparent to those skilled in the art that various modifications and variations can be made in the disclosed structures and methods without departing from the scope or spirit of the invention. Particularly, descriptions of any one embodiment can be freely combined with descriptions of other embodiments to result in combinations and/or variations of two or more elements and/or limitations. Other embodiments of the invention will be apparent to those skilled in the art from consideration of the specification and practice of the invention disclosed herein. It is intended that the specification and examples be considered exemplary only, with a true scope and spirit of the invention being indicated by the following claims.

What is claimed is:

1. A process for purifying greenhouse gases, the process comprising:
contacting a greenhouse gas stream comprising a contaminant with a solvent stream to at least partially reduce an amount of the contaminant and form a used solvent stream; and
using the greenhouse gas stream for carbon sequestration, enhanced oil recovery, industrial gas supply, chemical synthesis and production, or combinations thereof.

2. The process of claim 1, wherein the greenhouse gas stream comprises primarily carbon dioxide.

3. The process of claim 1, further comprising:
supplying the solvent stream for contacting the greenhouse gas stream from an acid gas removal unit; and
returning the solvent stream after contacting with the greenhouse gas stream to the acid gas removal unit.

4. The process of claim 1, wherein the solvent stream comprises primarily methanol.

5. The process of claim 1, wherein the solvent stream comprises a temperature of between about 20 degrees Celsius and about −80 degrees Celsius prior to contacting the greenhouse gas stream.

6. The process of claim 1, wherein a sulfur recovery unit tail gas stream supplies at least a portion of the greenhouse gas stream.

7. The process of claim 1, further comprising treating the greenhouse gas stream in a tail gas treatment unit before contacting with the solvent.

8. The process of claim 1, wherein the contaminant comprises primarily water.

9. The process of claim 1, wherein the greenhouse gas stream with a reduced amount of the contaminant comprises a dew point of between about 0 degrees Celsius and about −60 degrees Celsius,

10. An apparatus for purifying greenhouse gases, the apparatus comprising:
a greenhouse gas stream comprising a contaminant;
a solvent stream;
a contacting unit connected to the greenhouse gas stream and the solvent stream;
a contacting unit effluent stream connected to the contacting unit with a reduced amount of the contaminant; and
a used solvent stream connected to the contacting unit.

11. The apparatus of claim 10, wherein the contacting unit comprises mixing valves, static mixers, trays, random packing, structured packing, demisters, or combinations thereof.

12. The apparatus of claim 10, further comprising:
a sulfur recovery unit tail gas treatment unit connected to the greenhouse gas stream to supply at least a portion of the greenhouse gas stream;
an acid gas removal unit for supplying the solvent stream and receiving the used solvent stream; and
a recycle compression unit connected to the contacting unit effluent stream for carbon sequestration, enhanced oil recovery, industrial gas supply, chemical synthesis and production, or combinations thereof.

13. The apparatus of claim 10, further comprising a compression unit on the greenhouse gas stream or the contacting unit effluent stream, where the compression unit comprises a single stage device, a multistage device, or combinations thereof; and
optionally a booster compression device or pump on the green house gas stream.

14. The apparatus of 10, wherein:
the greenhouse gas stream comprises primarily carbon dioxide;
the solvent stream comprises primarily methanol; and
the contaminant comprises primarily water.

15. The apparatus of claim 10, wherein:
the solvent stream comprises a temperature of between about 20 degrees Celsius and about −80 degrees Celsius; and
the greenhouse gas effluent stream with a reduced amount of the contaminant after the contacting unit comprises a dew point of between about 0 degrees Celsius and about −60 degrees Celsius.

16. A process of drying carbon dioxide, the process comprising:
reacting a feedstock stream in a reactor unit to form a reactor unit effluent stream;
converting the reactor unit effluent stream in a shift conversion unit to form a shift conversion unit effluent stream;
separating the shift conversion unit effluent stream in an acid gas removal unit to form a hydrogen stream, a hydrogen sulfide stream, and a carbon dioxide stream;
recovering sulfur from the hydrogen sulfide stream in a sulfur recovery unit to form a sulfur stream and a sulfur recovery unit effluent stream;
contacting the sulfur recovery unit effluent stream in a tail gas treatment unit to form a tail gas treatment unit effluent stream comprising a reduced amount of hydrogen sulfide and a contaminant;
contacting the tail gas treatment unit effluent stream in a contacting unit with a solvent stream to form a used solvent stream and a purified tail gas treatment unit effluent stream comprising carbon dioxide and a reduced amount of the contaminant;
combining the carbon dioxide stream and the purified tail gas treatment unit effluent stream to form a combined carbon dioxide stream; and
using the combined carbon dioxide stream for carbon sequestration, enhanced oil recovery, industrial gas supply, chemical synthesis and production, or combinations thereof.

17. The process of claim 16, wherein the reactor unit comprises a gasification unit, a reforming unit, a partial oxidation unit, a pyrolysis unit, or combinations thereof.

18. The process of claim 16, further comprising using the hydrogen stream to produce steam, electricity, ammonia, methanol, synthetic hydrocarbon products, or combinations thereof.

19. The process of 16, wherein:
the greenhouse gas stream comprises primarily carbon dioxide;
the solvent stream comprises primarily methanol; and
the contaminant comprises primarily water.

20. The process of claim 16, wherein:
the solvent stream comprises a temperature of between about 20 degrees Celsius and about −80 degrees Celsius; and
the purified tail gas treatment unit effluent stream comprises a dew point of between about least 0 degrees Celsius and about −60 degrees Celsius.

21. An apparatus for producing syngas, the apparatus comprising:
a feedstock stream;
a reactor unit connected to the feedstock stream;
a reactor unit effluent stream connected to the reactor unit;
a shift conversion unit connected to the reactor unit effluent stream;
a shift conversion unit effluent stream connected to the shift conversion unit;
an acid gas removal unit connected to the shift conversion unit effluent stream;
a hydrogen stream connected to the acid gas removal unit;
a hydrogen sulfide stream connected to the acid gas removal unit;
a carbon dioxide stream connected to the acid gas removal unit;
a sulfur recovery unit connected to the hydrogen sulfide stream;
a sulfur stream connected to the sulfur recovery unit;
a sulfur recovery unit effluent stream connected to the sulfur recovery unit;
a tail gas treatment unit connected to the sulfur recovery unit effluent stream;
a tail gas treatment effluent stream comprising a contaminant connected to the tail gas treatment unit;
a solvent stream connected to the acid gas removal unit;
a contacting unit connected to the tail gas treatment unit effluent stream and the solvent stream to reduce an amount of the contaminant;
a contacting unit effluent stream connected to the contacting unit;
a used solvent stream connected to the contacting unit and the acid gas removal unit; and
a greenhouse gas unit connected to the contacting unit effluent stream for carbon sequestration, enhanced oil recovery, industrial gas supply, chemical synthesis and production, or combinations thereof.

22. The apparatus of claim 21, wherein the contacting unit comprises mixing valves, static mixers, trays, random packing, structured packing, demisters, or combinations thereof.

23. The apparatus of claim 21, further comprising a compression unit on the tail gas treatment unit effluent stream or the contacting unit effluent stream.

24. The apparatus of 21, wherein:
the tail gas treatment unit effluent stream comprises primarily carbon dioxide;
the solvent stream comprises primarily methanol; and
the contaminant comprises primarily water.

25. The apparatus of claim 21, wherein:
the solvent stream comprises a temperature of between about 20 degrees Celsius and bout −80 degrees Celsius; and
the contacting unit effluent stream comprises a dew point of between about 0 degrees Celsius and about −60 degrees Celsius.

26. The apparatus of claim 21, wherein the reactor unit comprises a gasification unit, a reforming unit, a partial oxidation unit, a pyrolysis unit, or combinations thereof.

27. The apparatus of claim 21, further comprising a steam generation unit, an electricity generation unit, an ammonia generation unit, a methanol generation unit, a synthetic hydrocarbon product generation unit, or combinations thereof.
